(19) United States
(12) Patent Application Publication
(10) Pub. No.: US 2010/0063289 A1
(43) Pub. Date: Mar. 11, 2010

(54) METHOD FOR PRODUCING AMMONIUM HEXAFLUOROPHOSPHATES

(76) Inventor: Joerg Brandenburg, Wiesbaden (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(21) Appl. No.: 12/523,553

(22) PCT Filed: Jan. 28, 2008
(86) PCT No.: PCT/EP2008/050987

§ 371 Date: Sep. 15, 2009

(30) Foreign Application Priority Data
Jan. 29, 2007 (EP) 07101364.3

(51) Int. Cl. C07D 211/74; C07D 451/06; C07D 491/18

(52) U.S. Cl. 546/91; 546/127; 546/89; 546/242

(57) ABSTRACT

The invention relates to a method for producing ammonium hexafluorophosphates of general formula (1) wherein R¹, R², R³ and R⁴ are defined as in the claims and in the description, said novel ammonium hexafluorophosphates and to the use thereof for producing pharmaceutically active compounds.

The invention relates to a method for producing ammonium hexafluorophosphates of general formula 1

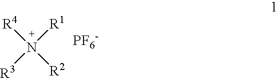

wherein R¹, R², R³ and R⁴ may have the meanings given in the claims and in the specification, new ammonium hexafluorophosphates as such and the use thereof for preparing pharmaceutically active compounds.

### DESCRIPTION OF THE INVENTION

The present invention relates to a method for producing ammonium hexafluorophosphates of formula 1

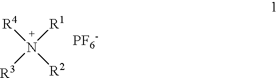

wherein
R¹ and R² which may be identical or different denote hydrogen or a group selected from among C₁-C₁₀-alkyl, C₂-C₁₀-alkenyl, C₂-C₁₀-alkynyl, C₃-C₈-cycloalkyl, C₄-C₈-cycloalkenyl, C₆-C₈-cycloalkynyl, C₆-C₁₀-aryl-C₁-C₆-alkyl, C₆-C₁₀-aryl-C₂-C₆-alkenyl, C₆-C₁₀-aryl-C₂-C₆-alkynyl, C₆-C₁₀-aryl and heterocyclyl, which may optionally be substituted;
R³ and R⁴ together with the nitrogen denote a mono-, bi- or tricyclic, saturated or unsaturated carbocyclic group which may contain 4 to 10 carbon centres, wherein optionally one or two of these carbon centres may be replaced by O or S, and which may optionally be substituted;
   characterised in that a compound of formula 2

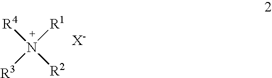

wherein R¹, R², R³ and R⁴ have the meanings given hereinbefore for compound 1 and wherein

X⁻ may denote an anion with a single negative charge,

is converted into the compound of formula 1 in a suitable solvent by reacting with a salt Kat⁺PF⁶⁻, where Kat⁺ denotes a cation selected from among Li⁺, Na⁺, K⁺, Mg2+, Ca2+,
with the proviso that the compound of formula 1 cannot be the compound of formula 1′

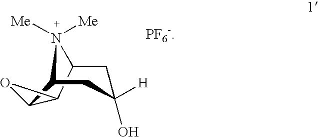

A particularly preferred process according to the invention is characterised in that the reaction of the compound of formula 2 to form the compound of formula 1 is carried out using a salt Kat⁺PF₆⁻, wherein Kat⁺ is selected from among Li⁺, Na⁺ and K⁺, particularly preferably Na⁺ and K⁺. Within the scope of the present invention the salts of the salt Kat⁺PF₆⁻ are optionally also referred to as salts of the salt KatPF₆.

The solvents used to carry out the process according to the invention are preferably polar solvents. Preferred solvents are selected according to the invention from among water, methanol, ethanol, propanol, isopropanol and mixtures thereof, while water, methanol and mixtures thereof are of exceptional importance according to the invention.

According to the invention preferably 1 mol, more preferably 1-1.5 mol, optionally also 2-5 mol of the salt KatPF₆ are used per mol of the compound of formula 2 used. It is apparent to the skilled man that the use of smaller amounts of salt KatPF₆ is possible, but that this may then lead to only a partial reaction of the compound of formula 2.

The process according to the invention is preferably carried out under mild reaction conditions, i.e. at temperatures in the range from 10-55° C., particularly preferably 15-50° C., particularly preferably 20-45° C. After all the salts KatPF₆ have been added, and to some extent even during their addition, the compounds of formula 1 crystallise out from the solution. The products obtained may, if necessary, be purified by recrystallisation from one of the above-mentioned solvents. The crystals obtained are isolated and dried in vacuo.

Preferred processes, according to the invention, for preparing the compounds of formula 1 are those wherein
R¹ and R² which may be identical or different denote hydrogen or a group selected from among C₁-C₆-alkyl, C₂-C₆-alkenyl, C₂-C₆-alkynyl, C₃-C₆-cycloalkyl, C₆-C₁₀-aryl-C₁-C₄-alkyl and C₆-C₁₀-aryl, which may optionally be substituted by one or more groups selected from among OH, F, Cl, Br, ═O, CN, NO₂, —C₁-C₄-alkoxy and —COOC₁-C₄-alkyl;
R³ and R⁴ together with the nitrogen denotes a mono-, bi- or tricyclic, saturated or unsaturated carbocyclic group which may contain 4 to 10 carbon centres, while optionally one or two of these carbon centres may be replaced by O, and which may optionally be substituted by one or more groups selected from among OH, F, Cl, Br, ═O, CN, NO₂, —C₁-C₄-alkoxy, C₁-C₄-alkyl, —COOC₁-C₄-alkyl, and —O—COR′, wherein
   R′ denotes a group selected from among C₁-C₄-alkyl, C₂-C₆-alkenyl and C₁-C₄-alkylene-phenyl, which may be substituted in each case by hydroxy, hydroxymethyl or C₁-C₄-alkoxy,
      with the proviso that the compound of formula 1 cannot be the compound of formula 1′

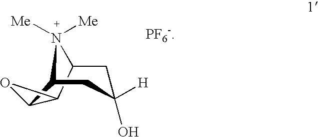

Also preferred, according to the invention, are processes for preparing the compounds of formula 1 wherein
R¹ and R² which may be identical or different denote hydrogen or a group selected from among C₁-C₆-alkyl, C₂-C₆-alkenyl, phenylethyl, benzyl and phenyl, which may optionally be substituted by one or more groups selected from among OH, F, Cl, Br, ═O, CN, NO₂, methoxy, ethoxy and —COOMe;
R³ and R⁴ together with the nitrogen form a group selected from pyrrole, pyrroline, pyrrolidine, pyridine, piperidine, morpholine,

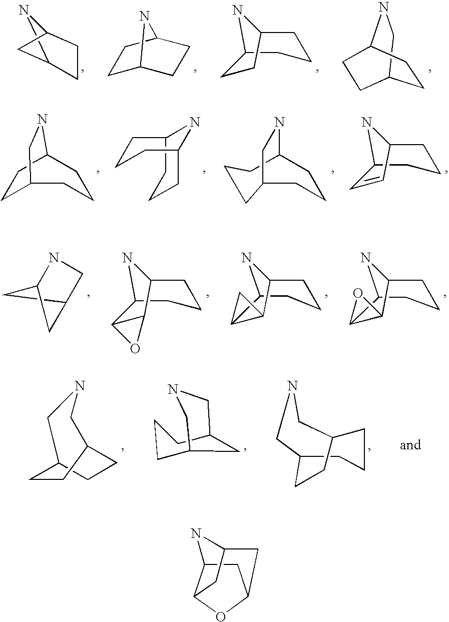

which may optionally be substituted by one or more, preferably one group selected from among OH, F, ═O, methyl, ethyl, methoxy and —O—COR′, wherein
R′ denotes a group selected from among C₁-C₄-alkyl, benzyl and phenylethyl, which may be substituted in each case by hydroxy, hydroxymethyl or methoxy;
   with the proviso that the compound of formula 1 cannot be the compound of formula 1′

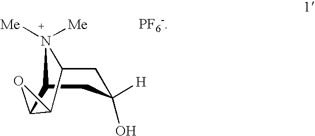

Also preferred, according to the invention, are processes for preparing the compounds of formula 1 wherein
R¹ and R² which may be identical or different denote hydrogen or a group selected from among C₁-C₄-alkyl, C₂-C₄-alkenyl, phenylethyl, benzyl and phenyl, which may optionally be substituted by one or more groups selected from among OH, F, Cl, Br, ═O, CN, NO₂, methoxy, ethoxy and —COOMe;
   and the groups R³ and R⁴ may have the meanings given hereinbefore or hereinafter.

Also preferred, according to the invention, are processes for preparing the compounds of formula 1 wherein
R¹ and R² which may be identical or different denote hydrogen or a group selected from among methyl, ethyl, propyl, butyl, benzyl and phenyl, which may optionally be substituted by one or more groups selected from among OH, F and ═O;
   and the groups R³ and R⁴ may have the meanings given hereinbefore or hereinafter.

Also preferred, according to the invention, are processes for preparing the compounds of formula 1 wherein R¹ denotes methyl and R² and the groups R³ and R⁴ may have the meanings given hereinbefore or hereinafter. Also particularly preferred according to the invention are processes for preparing the compounds of formula 1 wherein R¹ and R² represent methyl and the groups R³ and R⁴ may have the meanings given hereinbefore or hereinafter.

Also preferred, according to the invention, are processes for preparing the compounds of formula 1 wherein
R³ and R⁴ together with the nitrogen form a group selected from pyrrole, pyrroline, pyrrolidine, pyridine, piperidine, morpholine,

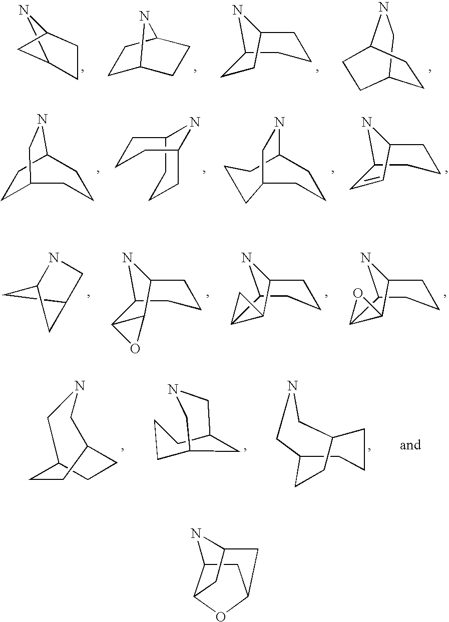

which may optionally be substituted by one or more, preferably one group selected from among OH, F, ═O, methyl, ethyl, methoxy and —O—COR′, wherein
R′ denotes a group selected from among C₁-C₄-alkyl, benzyl and phenylethyl, which may be substituted in each case by hydroxy, hydroxymethyl or methoxy;
   and the groups R¹ and R² may have the meanings given hereinbefore or hereinafter.

Also preferred, according to the invention, are processes for preparing the compounds of formula 1 wherein
R³ and R⁴ together with the nitrogen form a group selected from pyrroline, pyrrolidine, piperidine,

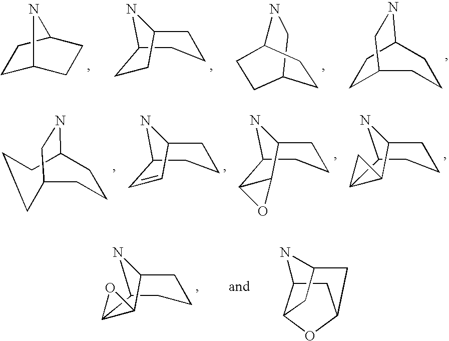

which may optionally be substituted by one or more, preferably one group selected from among OH, F, ═O, methyl, ethyl, methoxy and —O—COR′, wherein
R denotes a group selected from —CH₃, —CH₂—CH₃, —CH₂—CH₂—OH, —CH(OH)—CH₃, —CH₂-phenyl, —CH(OH)-phenyl and —CH(CH₂OH)-phenyl, preferably —CH₃, —CH₂—CH₃, —CH₂-phenyl, and —CH(CH₂OH)-phenyl, particularly preferably —CH(CH₂OH)-phenyl,
   and the groups R¹ and R² may have the meanings given hereinbefore or hereinafter.

Also preferred, according to the invention, are processes for preparing the compounds of formula 1 wherein
R³ and R⁴ together with the nitrogen form a group selected from pyrrolidine, piperidine,

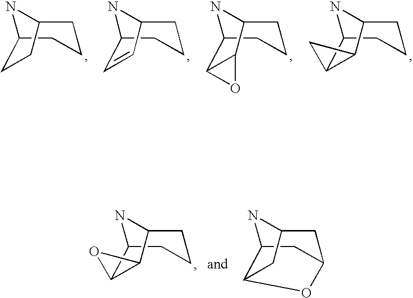

which may optionally be substituted by one or more, preferably one group selected from among OH, F, ═O, methyl, ethyl, methoxy and —O—COR′,
wherein
R denotes a group selected from —CH₃, —CH₂—CH₃, —CH₂—CH₂—OH, —CH(OH)—CH₃, —CH₂-phenyl, —CH(OH)-phenyl and —CH(CH₂OH)-phenyl, preferably —CH₃, —CH₂—CH₃, —CH₂-phenyl, and —CH(CH₂OH)-phenyl, particularly preferably —CH(CH₂OH)-phenyl,
   and the groups R¹ and R² may have the meanings given hereinbefore or hereinafter.

Examples of alkyl groups, as well as alkyl groups which are a part of other groups, include to branched and unbranched alkyl groups with 1 to 10 carbon atoms. These include: methyl, ethyl, propyl, butyl. Unless stated otherwise, the above-mentioned designations propyl and butyl include all the possible isomeric forms. For example, the term propyl includes the two isomeric groups n-propyl and iso-propyl, the term butyl includes n-butyl, iso-butyl, sec. butyl and tert.-butyl.

Examples of alkoxy or alkyloxy groups are branched and unbranched alkyl groups with 1 to 10 carbon atoms which are linked by an oxygen atom. These include: methoxy, ethoxy, propoxy, butoxy. Unless stated otherwise, the above-mentioned designations include all the possible isomeric forms.

Examples of alkenyl groups as well as alkenyl groups which are part of other groups are branched and unbranched alkyl groups with 1 to 10 carbon atoms, provided that they contain at least one double bond.

Examples of alkynyl groups as well as alkynyl groups which are part of other groups are branched and unbranched alkyl groups with 1 to 10 carbon atoms, provided that they contain at least one triple bond.

Examples of cycloalkyl groups with 3-8 carbon atoms are cyclic alkyl groups such as for example cyclopropyl, cyclobutyl, cyclopentyl or cyclohexyl.

Examples of cycloalkenyl groups with 4-8 carbon atoms are cycloalkyl groups, provided that they contain at least one double bond.

Examples of cycloalkenyl groups with 6-8 carbon atoms are cycloalkyl groups, provided that they contain at least one triple bond.

Examples of aryl groups are aromatic ring systems with 6 to 10 carbon atoms. Preferred aryl groups are phenyl and naphthyl, while phenyl is of particular importance.

Aryl-alkyl groups are aryl groups that are linked via alkyl groups. Preferred arylalkyl groups are phenylethyl and benzyl.

Aryl-alkenyl groups are aryl groups that are linked via alkenyl groups.

Aryl-alkynyl groups are aryl groups that are linked via alkynyl groups.

Heterocyclyl groups are 5-, 6- or 7-membered, saturated or unsaturated heterocycles which may contain nitrogen, oxygen or sulphur as heteroatoms. Examples include furan, tetrahydrofuran, tetrahydrofuranone, γ-butyrolactone, α-pyran, γ-pyran, dioxolan, tetrahydropyran, dioxane, thiophene, dihydrothiophene, thiolan, dithiolan, pyrrole, pyrroline, pyrrolidine, pyrazole, pyrazoline, pyrazolidine, imidazole, imidazoline, imidazolidine, triazole, tetrazole, pyridine, piperidine, pyridazine, pyrimidine, pyrazine, piperazine, triazine, tetrazine, morpholine, thiomorpholine, diazepan, oxazole, isoxazole, oxazine, thiazole, isothiazole, thiadiazole, oxadiazole, pyrazolidine.

The group ═O denotes a carbonyl group. The group —O—CO—R′ denotes an ester function.

The salts of quaternary ammonium compounds, such as for example those of formula 2, are generally readily soluble in water and alcohol. However, they are extremely poorly soluble in less polar organic solvents such as for example acetone, acetonitrile, hydrocarbons, halohydrocarbons or ethers. Chemical reactions with quaternary ammonium compounds are therefore limited in principle to reactions in water, alcohol or strongly polar aprotic solvents such as DMF (dimethylformamide) or NMP (N-methylpyrrolidine). This gives rise to severe restrictions as to the choice of reactants or their separation from the target product.

Many synthesis strategies fail as a result of the impossibility or difficulty of separating quaternary ammonium compounds in aqueous or alcoholic solutions from other reaction components. This problem can be solved using the ammonium ions of formula 1. The selective precipitation or crystallisation of the quaternary ammonium compounds of formula 1 from alcohols or water may be carried out by reacting the compounds 2 with the corresponding salts KatPF₆ and in this way they can be isolated and purified with a regularly high yield.

By virtue of their very good solubility and the exceptionally high stability of the anion, the compounds 1 make it possible to carry out a range of reactions in less polar aprotic solvents and may be used wherever water or alcohol creates a problem. Because of these properties the compounds of formula 1 are valuable starting materials in the synthesis of modified quaternary ammonium salts in organic solvents. After the hexafluorophosphates have been reacted to form the desired modified ammonium hexafluorophosphates, the hexafluorophosphate can be replaced again by other anions using lithium salts (such as e.g. LiBr) for example. The synthesis of tiotropium salts described in the experimental section of the present invention may serve as an example of this.

Against this background, the present invention further relates to ammonium hexafluorophosphates of general formula 1 as such,

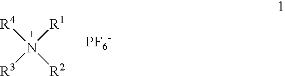

wherein R¹, R², R³ and R⁴ may have the above-mentioned meanings, with the proviso that the compound of formula 1 cannot be tiotropium hexafluorophosphate or the compound of formula 1′

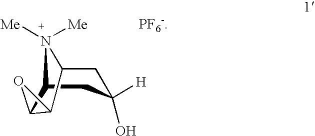

In another aspect the present invention relates to the use of the above-mentioned hexafluorophosphates of formula 1 as starting compounds for preparing ammonium salts.

The following Examples serve to illustrate methods of synthesis carried out by way of example. They are intended purely as possible methods described by way of example without restricting the invention to their content.

### EXAMPLES OF SYNTHESIS—GENERAL PROCEDURE

An ammonium compound of formula 2 is dissolved in water and combined with an equimolar amount or molar excess of a water-soluble hexafluorophosphate (sodium or potassium salt).

The hexafluorophosphate of formula 1 is precipitated or crystallised as a white water-insoluble product, then isolated, optionally washed with methanol and then dried at approx. 40° C. in the drying cupboard.

The following compounds were obtained analogously to the general procedure described above.

### Example 1

### N-methyltropinium hexafluorophosphate

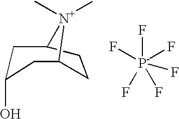

18.4 g of N-methyltropinium iodide are dissolved in 50 ml of water and brought to crystallisation by the addition of a solution of 11.4 g NaPF6 30 ml of water. The crystals are filtered, washed with water and dried. Yield: 19.6 g (74%)

### Example 2

### 4-hydroxy-6.6-dimethyl-2-oxa-6-azonium-tricyclo[3.3.1.0*3,7*]nonane hexafluorophosphate

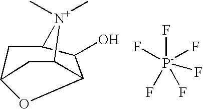

4-hydroxy-6.6-dimethyl-2-oxa-6-azonium-tricyclo[3.3.1.0*3.7*]nonane bromide (20 g) is dissolved in methanol (100 ml), brought to reaction (rearrangement) with the addition of a catalytic amount (4-14 mol %) of sodium methoxide at reflux temperature and then combined with an equimolar amount or molar excess of a solution of sodium hexafluorophosphate (13 g) in 33 ml of methanol.

The 4-hydroxy-6,6-dimethyl-2-oxa-6-azonium-tricyclo[13.3.1.0*3,7*]nonane hexafluorophosphate is precipitated/crystallised as a white, poorly water-soluble product, which is isolated, optionally washed with methanol and then dried at approx. 40° C. in vacuo. Yield: 25 g (72%); m.p: 292-293° C.

### Example 3

### 8.8-dimethyl-3-oxo-8-azonium-bicyclo[3.2.1]oct-6-ene hexafluorophosphate

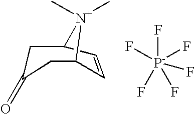

1 g of 8,8-dimethyl-3-oxo-8-azonium-bicyclo[3.2.1]oct-6-ene bromide are dissolved in 25 ml of water and brought to crystallisation by the addition of a solution of 0.62 g sodium hexafluorophosphate in 10 ml of water. The crystals are filtered, washed with water and dried. Yield: 1.3 g

### Example 4

### 1,1-dimethyl-4-oxo-piperidinium hexafluorophosphate

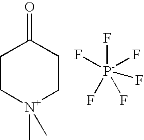

6.7 g of 1,1-dimethyl-4-oxo-piperidinium bromide are dissolved in 30 ml of water and brought to crystallisation by the addition of a solution of 5.9 g sodium hexafluorophosphate in 30 ml of water. The crystals are filtered, washed with water and dried. Yield: 8.8 g (57%); M.p.: 220-221° C.

### Example 5

### N-methylscopolaminium hexafluorophosphate

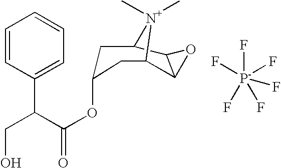

20 g N-methylscopolaminium bromide are dissolved in 200 ml of water and combined with a solution of 9.2 g sodium hexafluorophosphate in 50 ml of water (25° C.). The crystals precipitated are washed with 50 ml of water and dried. Yield: 23.3 g (83%)

The following synthesis examples show that the use of hexafluorophosphates of formula 1 allows syntheses with ammonium compounds to be carried out easily.

### Example 6

### 7-hydroxy-9,9-dimethyl-3-oxa-9-azonium-tricyclo[3.3.1.0*2,4*]nonane hexafluorophosphate

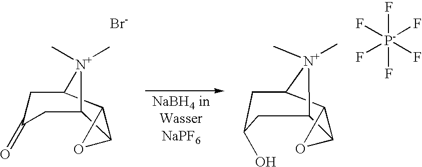

3.3 g 9,9-dimethyl-7-oxo-3-oxa-9-azonium-tricyclo[3.3.1.0*2,4*]nonane bromide were dissolved in 33 ml of water and within 1 hour combined with 1 g NaBH₄ and some HCOOH while being cooled. After the reaction was complete, 2.5 g NaPF₆ were added. The crystals precipitated are suction filtered, washed and dried. Yield: 4.2 g (43%)

As the hexafluorophosphate is removed from the equilibrium by crystallisation, this reaction is easy to carry out.

The following Example serves to illustrate how hexafluorophosphates of formula 1 which are poorly soluble in water can easily be converted into water-soluble salts.

### Example 7

### Re-conversion into the Water-Soluble Salt

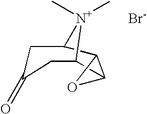

5.5 g of 9,9-dimethyl-7-oxo-3-oxa-9-azonium-tricyclo[3.3.1.0*2,4*]nonane hexafluorophosphate are dissolved in 50 ml acetone and combined with a solution of 1.8 g LiBr in 20 ml acetone. The crystals precipitated are suction filtered, washed and dried. Yield 4.4 g (85%); M.p. 200-202° C.;

Using tiotropium bromide as an example, the following Examples illustrate how complex pharmaceutical active substances can be obtained by a gentle procedure under simplified conditions using the hexafluorophosphates according to the invention.

### Example 8

### N-methylscopinium hexafluorophosphate

N-methylscopinium bromide is dissolved in water and combined with an equimolar amount or molar excess of a water-soluble hexafluorophosphate (sodium or potassium salt). Aqueous hexafluorophosphoric acid also leads to precipitation.

The N-methylscopinium-hexafluorophosphate is precipitated/crystallised as a white, water-insoluble product, which is isolated, optionally washed with methanol and then dried at approx. 40° C. in the drying cupboard.

M.p.: 265-267° C. (melts with discoloration);

H-NMR: in acetonitrile-d3 σ(ppm): 1.9 (dd, 2H), 2.55(dd, 2H), 2.9 (s.3H), 3.29 (s. 3H), 3.95(dd, 4H), 3.85 (s, 1H).

### Example 9

### Tiotropium bromide

1.6 g (5 mmol) of methylscopinium hexafluorophosphate (Example 10) and 2.0 g (7.8 mmol) of methyl dithienylglycolate are refluxed in 50 ml acetone and in the presence of 10 g of molecular sieve 4A for 50-70 hours.

The reaction mixture is filtered, the filtrate is combined with a solution of 0.3 g of LiBr in 10 ml of acetone. The still unreacted N-methylscopinium bromide that has crystallised out is separated off by filtration. After the addition of another 0.6 g LiBr (dissolved in acetone) tiotropium bromide is precipitated in an isolated yield of 30% (based on the compound of Example 9 used).

### Example 10

### Tiotropium hexafluorophosphate

Tiotropium hexafluorophosphate is not isolated within the scope of the reaction according to Example 10 but further reacted directly to form the tiotropium bromide.

For the purposes of characterising the tiotropium hexafluorophosphate this was prepared specifically and isolated. The following characterising data were obtained.

M.p.: 233-236° C. (melting with discoloration)

H-NMR: in acetone-d6: σ(ppm): 2.08 (dd, 2H), 2.23(dd, 2H), 3.32 (s. 3H), 3.50 (s, 3H), 3.62(s, 2H), 4.28(m, 2H), 5.39(m, 1H) .6.25 (s), 7.02(m, 2H), 7.027.22(m, 2H), 7.46(m, 2H), P-NMR: in acetone-d6: σ(ppm): −143.04, heptet, J=4.37.

### Example 11

### Tiotropium bromide

31.5 g (100 mmol) methylscopinium hexafluorophosphate (Example 9) and 25.4 g (100 mmol) methyl dithienylglycolate are refluxed in 400 ml acetone and in the presence of 40 g of powdered molecular sieve 4A (Fluka) and DMAP (4,4-dimethylaminopyridine) for 24 h. (Molecular sieve was replaced by the same amount after 3 h.)

The reaction mixture is filtered, washed with 200 ml acetone, the filtrate is combined stepwise with a solution of 9.6 g LiBr (110 mmol) in 110 ml acetone. The unreacted N-methylscopinium bromide that crystallises out is separated off by filtration. (Fractionated precipitation). The crystal fractions were filtered off and dried. The composition of the fractions was determined by thin layer chromatography. Tiotropium bromide in an isolated yield of 16.6 g (35%) (based on the compound of Example 9 used). Purity HPLC>99%. Purity according to TLC: no impurities could be detected.

### Example 12

### Tiotropium bromide

1.6 g (5 mmol) of methylscopinium hexafluorophosphate (Example 9) and 1.25 g (5 mmol) of methyl dithienylglycolate are stirred in 50 ml acetone and in the presence of 2 g of powdered molecular sieve 4A (Fluka) and 6 mg of potassium-tert.-butoxide at 0° C. for 4 h. The reaction mixture is filtered, washed with 20 ml acetone, the filtrate is combined stepwise with a solution of 0.7 g LiBr (13 mmol) in 11 ml acetone. The unreacted that crystallises out is separated off by filtration. (Fractionated precipitation). The crystal fractions were filtered off and dried. The composition of the fractions was determined by thin layer chromatography. The tiotropium bromide fractions were suction filtered, washed with acetone, recrystallised from water, washed with acetone and dried. 1.2 g (48% based on the compound of Example 9 used) were isolated in this way. Purity HPLC: 99.8%. Purity according to TLC: no impurities were visible.

### Example 13

### Tiotropium bromide

31.5 g (0.1 mol) methylscopinium hexafluorophosphate (Example 9) and 30.5 g (0.10 mol) of 2,2′-methyl dithienylglycolate are dissolved in 400 ml acetone and stirred in the presence of 90 g of zeolite of type 4A (Na₁₂Al₁₂Si₁₂O₄₈×n H₂O) and 0.2 g (1 mmol) potassium-tert.-butoxide over a period of 20-24 hours at 0° C.

The reaction mixture is filtered, the filtrate is combined with a solution of 8.7 g of LiBr (8.7 g 0.10 mol in 100 ml acetone).

The product that crystallises out is separated off by filtration, washed with acetone and then dried.

A yield of 41.4 g (87.7%) is obtained, with a 90% conversion level.

The reactions described by way of example take place with virtually no by-products being formed. In optional cases where the reactions are supposed to take place without total reaction of the starting materials, the N-methylscopinium bromide isolated in the first step of working up is therefore recycled using the reaction according to Example 10 and in this way the overall yield can be increased significantly within the scope of a production process.

1) Process for preparing ammonium hexafluorophosphates of formula 1 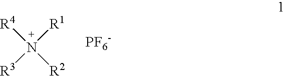
wherein
R¹ and R² which may be identical or different denote hydrogen or a group selected from among C₁-C₁₀-alkyl, C₂-C₁₀-alkenyl, C₂-C₁₀-alkynyl, C₃-C₈-cycloalkyl, C₄-C₈-cycloalkenyl, C₆-C₈-cycloalkynyl, C₆-C₁₀-aryl-C₁-C₆-alkyl, C₆-C₁₀-aryl-C₂-C₆-alkenyl, C₆-C₁₀-aryl-C₂-C₆-alkynyl, C₆-C₁₀-aryl and heterocyclyl, which may optionally be substituted;
R³ and R⁴ together with the nitrogen denote a mono-, bi- or tricyclic, saturated or unsaturated carbocyclic group which may contain 4 to 10 carbon centres, wherein optionally one or two of these carbon centres may be replaced by O or S, and which may optionally be substituted;
characterised in that a compound of formula 2 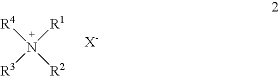
wherein R¹, R², R³ and R⁴ have the meanings given hereinbefore for compound 1 and wherein
X⁻ may denote an anion with a single negative charge,
is converted into the compound of formula 1 in a suitable solvent, by reacting with a salt Kat⁺PF⁶⁻, wherein Kat⁺ denotes a cation selected from among Li⁺, Na⁺, K⁺, Mg2+, Ca2+,
with the proviso that the compound of formula 1 may not be the compound of formula 1′

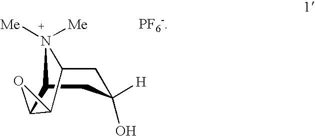

2) Process according to claim 1, characterised in that it is carried out with a salt Kat⁺PF₆⁻, wherein Kat⁺ is selected from among Li⁺, Na⁺ and K⁺, particularly preferably Na⁺ and K⁺.

3) Process according to claim 1, characterised in that the reaction is carried out in a polar solvent, preferably in a solvent selected from among water, methanol, ethanol, propanol and isopropanol, preferably water or methanol or mixtures thereof.

4) Ammonium hexafluorophosphates of general formula 1, 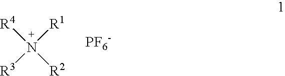
wherein R¹, R², R³ and R⁴ may have the meanings given in claim 1, with the proviso that the compound of formula 1 may not be tiotropium hexafluorophosphate or the compound of formula 1′ [chemical structure]
